(19) United States
(12) Patent Application Publication
(10) Pub. No.: US 2008/0031928 A1
(43) Pub. Date: Feb. 7, 2008

(54) NO LAXATION BULKING SYSTEM

(75) Inventors: George E. Steele, Fort Worth, TX (US); Donna Henry, Paris, TX (US); William E. Steele IV, Fort Worth, TX (US)

Correspondence Address: FULBRIGHT & JAWORSKI L.L.P, 2200 ROSS AVENUE, SUITE 2800, DALLAS, TX, 75201-2784, US

(73) Assignee: Remington Direct LP, Fort Worth, TX (US)

(21) Appl. No.: 11/828,950

(22) Filed: Jul. 26, 2007

Related U.S. Application Data
(60) Provisional application No. 60/833,551, filed on Jul. 26, 2006.

(51) Int. Cl. A23L 1/05; A21D 10/00; A23L 1/22; A61K 9/68

(52) U.S. Cl. 424/440; 426/533; 426/549; 426/576

(57) ABSTRACT

A no-laxation bulking system and a method for preparation of same is disclosed. Gelatin and pectin are combined to fro a dry-blended mixture. A second set of ingredients comprising modified polydextrose and erithritol is then blended to form a first liquid mixture. A second liquid mixture comprising at least one acid and water is formed. The dry-blended mixture is added to the first liquid mixture, forming a first combination. Water is added to the first combination, and the resultant combination is heated to a first predetermined temperature. The first predetermined temperature is maintained for a predetermined time period, and the heat is reduced to a second predetermined temperature. The second liquid mixture is added to the combination. Other embodiments comprise adding hydrogenated starch hydrolysate (HSH) and/or active ingredients. The second liquid mixture also may preferably include fruit concentrate, flavor, color and/or sucralose.

### CROSS-REFERENCE TO RELATED APPLICATIONS

The present application claims priority to co-pending U.S. Provisional Patent Application No. 60/833,551, entitled “No Laxation Bulking System,” filed Jul. 26, 2006, the disclosure of which is hereby incorporated herein by reference.

### TECHNICAL FIELD

The present invention relates generally to no laxation bulking systems and more particularly, to a system and method of forming a no laxation bulking system in the form of a soft candy or hard candy product for human ingestion.

### BACKGROUND OF THE INVENTION

In recent years, controlling sugar, calories, fat and carbohydrates in the human diet has become more of a concern. Many humans desire to control these items to satisfy special dietary needs, i.e., individuals suffering from diabetes or other health problems. Other humans are attempting to eat more healthfully as a preventative measure and/or so as to address weight loss or maintenance concerns.

Regardless what type of dietary needs a person has, typically snack foods are part of such diet. For example, those attempting to lose weight may ingest a number of small meals during the day, some of those meals being snack foods. Similarly, diabetics may require snack foods during the day in order to maintain a proper blood sugar level. Snack foods also may be eaten in order to provide energy or to deliver vitamins to the body. Thus, most humans incorporate snack foods into their diet, but the desire is to have a more healthful snack food. To prevent hunger, there exists a need for a food item to act as a low calorie stomach fill; this food item should be able to be consumed in large quantities to satiate hunger with no resultant weight gain.

Many consider confectionery items, such as candies, to be snack foods. In the past, candies have not been considered to be very healthy. In recent years, however, many confectioners begun developing ways to make more healthful confectionery items, such as sugar-free candies. The confectionery industry has been developed around the properties of one ingredient—sucrose—and thus, when developing sugar-free candies, the desire is to find substitutes for sucrose that most closely mirror the properties particularly the taste, of sucrose.

Polyols are often used as substitutes for sucrose in the manufacture of sugar-free confectionery items. Polyols typically used in sugar-free confectionery items include sorbitol, maltitol, lactitol, isomalt and polydextrose. Maltitol, which is produced by hydrogenating maltose in the form of a pure maltose glucose syrup, is believed to most closely resemble the properties of sucrose in that, like sucrose, maltitol is a disaccharide. However, the use of polyols, particularly maltitol, in these sugar-free confectionery items can often be disadvantageous due to the laxative effects that these polyols produce. The laxative effect is osmotic in origin given that the unabsorbed material upsets the osmotic balance within the intestinal system, and the consequences can be unpleasant for many who ingest sugar-free confectionery items containing such polyols. Thus, there exists a need for a bulking system, such as in a soft candy product, that is sugar-free while avoiding the laxation effects typically encountered with food items that incorporate polyols.

Sugar-free confectionery products are typically manufactured in a manner similar to that of sugar-containing confectionery products. In the case of soft candy products, the amount of time needed for the products to set prior to packaging and distribution is significant given that the formulation takes a long time to dry and cool. For example, when a confectionery item containing maltitol syrup is formed, typically it takes at least one to two days before the confectionery item has sufficiently dried and cooled. Thus, there exists a need for a method of forming a bulking system that sets, dries and cools in a shorter period of time.

### BRIEF SUMMARY OF THE INVENTION

The present invention is directed to a method for producing a no-laxation bulking system. A first set of ingredients comprising a gelatin and a pectin is blended. A second set of ingredients comprising polydextrose (or modified polydextrose) and erithritol is then blended. The next step includes blending a third set of ingredients comprising at least one acid and water. The first set of ingredients is added to the second set of ingredients, forming a first mix. Water is then added to the first mix, and the combination is heated to a first predetermined temperature. The first predetermined temperature is maintained for a predetermined time period, and then the heat is reduced to a second predetermined temperature. The third set of ingredients is then added to the combination. Embodiments of the invention further comprise adding hydrogenated starch hydrolysate (HSH) and/or active ingredients as components of the no-laxation bulking system. The third set of ingredients also may preferably include flavors, colors and/or sucralose or another high intensity sweetener.

Another embodiment of the invention is a no-laxation bulking system. The bulking system is preferably comprised of a dry-blended mixture comprising gelatin and pectin, a first liquid mixture of polydextrose (or modified polydextrose) and erithritol, wherein the dry-blended mixture is added to the first liquid mixture forming a first combination, without blending, and then water is introduced prior to heating the first combination. A second liquid mixture of at least one acid and water is formed, and the second liquid mixture is added to the first combination.

Further embodiments of the present invention are no-laxation soft and hard candy products. The soft or hard candy product preferably is comprised of a dry-blended mixture, wherein dry-blended mixture comprises 0 to 12% gelatin and 0 to 5% pectin. The soft or hard candy product is further comprised of a first liquid mixture, wherein the first liquid mixture is comprised of 10 to 80% polydextrose (or modified polydextrose) and 2 to 12% erithritol. A second liquid mixture further comprises the soft or hard candy product, wherein the second liquid mixture comprises 0 to 5% acid as well as water. The dry-blended mixture and the first liquid mixture are preferably combined. Water is added to the combination, and heat is applied until the temperature reaches a first predetermined temperature. After a set amount of time at the first predetermined temperature, the heat is reduced and the second liquid mixture is preferably added.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### DETAILED DESCRIPTION OF THE INVENTION

The desire is to achieve a stable gelatin matrix using a bulking agent. This is achieved by using a sugar alcohol such as erithritol as well as a polydextrose (or modified polydextrose), such as Litesse Ultra liquid, having high fiber content. The finished product is preferably zero sugar and low calorie providing for no laxation upon ingestion. A no-laxation bulking system, such as a soft or hard candy product, is preferably formed using a dry-blended mixture of pectin and gelatin added to a first liquid mixture of polydextrose (or modified polydextrose) and erithritol. Water is added to the combination of the dry-blended mixture and the first liquid mixture, and preferably the combination is stirred constantly until any lumps that may be present in the combination are dispersed. Heat is then applied to the solution until a first predetermined temperature level is reached. Preferably, this predetermined temperature is approximately 160 degrees Fahrenheit, although the predetermined temperature may vary depending on the scaling of the manufacturing process. The temperature is maintained at this predetermined level for a specified amount of time, and then the heat is reduced to a second predetermined temperature. This second predetermined temperature is preferably a low temperature, such as 100 to 110 degrees Fahrenheit, although again the temperature may vary depending on the scaling of the manufacturing process. Once the solution has reached the second predetermined temperature range, a second liquid mixture comprised of at least an acid and water is added to the solution. The resultant solution is then preferably stirred constantly and held in the kettle or mixing tank for panning or molding as desired. The product also may be extruded. Plating or molding is then conducted into sprayed or prepared pans. The pans are allowed to cool, preferably for one to two hours, and then the resulting product is depanned.

Gelatins are often used for making confectionery items. Gelatins produced by acid hydrolysis are referred to as type A, whereas gelatins produced by alkaline hydrolysis are typically referred to as type B. The pH of type A gelatin is typically higher than the pH of type B gelatin (6.3-9.5 versus 4.5-5.2). Gelatins are typically not stable in acid, and thus any addition of acid should be as late as possible in the manufacturing process. Preferably, a type B gelatin is used as part of the dry-blended mixture contemplated by the present invention. Specifically, a gelatin type B 250 bloom is preferred. This gelatin has been selected based on having the specific ability to allow gelatin to set at a lower temperature (i.e., reduce the amount of time for setting as well as the temperature at which setting occurs) when used in combination with other ingredients as described and to produce a firm soft or hard candy product.

Pectins also are often used for making confectionery items. However, typically they have not been used in conjunction with gelatins. This is due to the problem that if the pH does not fall within a specific range, the system formed may be unstable and may not set properly. However, in the present invention, pectin amid AF 020 is a preferable pectin because of its specific ability to work with gelatin in setting the bulking system without resulting in stickiness of the product when placed in combination with sugar alcohols or polyols.

In the first step of the bulking system manufacturing process, gelatin and pectin are dry-blended to form a mixture. This mixture is typically formed using a blender run at a slow speed for a specified period of time. Gelatin and pectin are combined through blending at a slow speed until even dispersement is achieved. No heat should be applied to the gelatin/pectin combination. The bulking system is typically comprised of 0 to 12% gelatin and 0 to 5% pectin. Preferably, however, the gelatin comprises 7 to 8% of the bulking system and pectin comprises less than 1% of the resultant bulking system. When such a composition is present, approximately two minutes is needed to form the dry-blended mixture such that the gelatin and pectin are evenly dispersed. However, as the amounts and percentages of gelatin and/or pectin change due to scaling of the manufacturing process, it should be appreciated that the time required to fully blend the pectin and gelatin also may change.

In the second step of the bulking system manufacturing process, polydextrose (or modified polydextrose) and erithritol are combined to form a first liquid mixture. A preferred polydextrose for use is Litesse. Litesse is known as a specialty carbohydrate (polydextrose) that replaces sugar and fat while improving flavor, texture and mouthfeel in a variety of applications. It is low glycemic and thus suitable for consumers seeking low impact carbohydrates. Further, Litesse is water soluble and is used as a bulking agent to make a variety of items lower in fat calories and sugar-free while also high in fiber and having a good taste. There are different types of Litesse that may preferably be utilized in forming of the bulking system of the present invention—Litesse Ultra liquid or a refined polydextrose FCC. While different forms of Litesse may be utilized, it may be preferable to use the liquid formulation (Litesse Ultra liquid) because there is typically less water present in the formulation, and it presents a clean taste with mildly sweet flavor. The percentage of polydextrose (or modified polydextrose) preferably present in the formulation can range from 10 to 80% of the resultant bulking system; however, it is preferable to employ a range of 45 to 65% in order to produce a settable soft or hard candy product. It should be appreciated that lower percentages of Litesse Ultra liquid may be used if another form of Litesse (such as Litesse II) is also used in the composition and/or a hydrogenated starch hydrolysate (HSH) also is employed.

Erithritol is a polyol that acts as a novel bulk sweetener and has a caloric value close to zero. It is the only polyol presently known that has been shown not to cause laxation effects when incorporated into sugar-free edible items. Erithritol is believed to avoid this laxative effect because it is excreted through the kidneys, and as it has a low molecular weight, more than 90% is absorbed in the small intestine. This fraction is not metabolized and is excreted unchanged through the urine. Thus, it is shown to have the highest digestive tolerance of the polyols as studies have shown that adults ingesting up to 1 g of erithritol per day per kilogram of body weight do not show any gastrointestinal effects.

Erithritol granules are preferably used in the bulking system contemplated by the present invention. Granules, as opposed to a powder for, are used as the powder version typically does not retain enough water for absorption. This lack of water retention may affect the process to the extent that setting of the bulking system may be hindered or even prevented. The bulking system preferably is comprised of a range of 2 to 12% of erithritol. However, it should be appreciated that under certain conditions, erithritol may crystallize and lose some of its effectiveness as a sugar substitute if used at levels comprising over 8% of the bulking system, particularly if the pH of the resulting product is low.

When polydextrose (or modified polydextrose) and erithritol granules are preferably combined, the mixing occurs using a Lightning mixer or a similar type mixer at a slow to medium speed. The amount of time for mixing will vary, but mixing should be continued until any lumps present in the combination are evenly dispersed. Mixing preferably will be completed after approximately five minutes, but again, it should be appreciated that as the manufacturing process is altered to employ more or less polydextrose (or modified polydextrose) and/or erithritol, the amount of time needed for mixing may be altered accordingly. It should be appreciated that the polydextrose (or modified polydextrose)/erithritol combination may preferably be mixed in the mixing kettle or tank ultimately used to combine all of the elements comprising the soft or hard candy resultant bulking system, although if preferred, this first liquid mixture may be transferred to the mixing kettle or tank at a later time following initial formation.

A second liquid mixture is formed by mixing at least one acid and a small amount of water together. Different acids may be utilized depending on the type of finished product desired. Malic acid is preferably used when a fruit-flavored product is formulated. Malic acid has characteristics that allow for setting the bulking system at a low temperature of approximately 155-160 degrees Fahrenheit. If a brown-flavored bulking system is desirable, lactic acid is typically used. Lactic acid has a desirable flavor profile and acts as a good setting acid for a chocolate-flavored, vanilla-flavored, mocha-flavored, cinnamon-flavored or cream/milk-flavored bulking systems, for example, at a low temperature such as 155-160 degrees Fahrenheit. It should be appreciated that the amount of acid used in the second liquid mixture may comprise anywhere from 0 to 5% of the resultant bulking system. Preferably, the acid comprises 1 to 2% of the bulking system.

Although only a combination of at least one acid and water is required to form the second liquid mixture, it should be appreciated that at this step, color, flavors and sucralose or some other high intensity sweetener may be blended with at least one acid and water to form the second liquid mixture. Preferred flavors may be fruit flavors or brown flavors, although other flavors may be added without departing front the object of the invention. Ascorbic acid (Vitamin C) may be used as a flavor enhancer as well as to provide Vitamin C content. However, whatever flavors utilized in forming the second liquid mixture are preferably concentrated and are in liquid form the majority of the time. Preferably the flavor level is at 1.75% or below. It should be appreciated that when concentrations of the flavor fall below this percentage, many off flavors begin to show up in the finished bulking system due to the carriers in the flavors and the reduction of the other ingredients to make a place for the carriers for flavors. For added flavor in fruit-flavored bulking systems, a fruit concentrate with essence returned is preferably utilized. For example, strawberry fruit puree concentrate with essence returned is a preferable fruit concentrate as it is low in calories and is 28 degree brix. It also has the essence returned allowing more flavor for a smaller amount of usage. Other fruit concentrates with essence returned may be utilized without departing from the objects of the present invention such as pear puree concentrate. A fruit concentrate typically comprises less than 1% of the resultant bulking system. Sucralose (such as Splenda) is preferably used for additional flavoring and comprises less than 1% of the bulking system similar to the fruit concentrate. Various colors can be used depending on the color desired for the bulking system, and the color typically comprises less than 0.01% of the bulking system. When at least one acid, color, flavors, sucralose, water and fruit concentrate are combined to preferably form the second liquid mixture, the components are combined until mixed and the color is well dispersed.

After the second liquid mixture is formed, the dry-blended mixture is added to the first liquid mixture. If the first liquid mixture comprised of polydextrose (or modified polydextrose) and erithritol is initially prepared in the mixing kettle or tank, then once the dry-blended mixture and the first liquid mixture are combined, water should be metered into the tank or mixing kettle. Alternatively, the amount of water to be added to the combination of the dry-blended mixture and the first liquid mixture may be weighed into a separate container and reserved for later addition to the combination.

The addition of the dry-blended mixture to the first liquid mixture is quickly followed by the addition of water. The resulting solution is then briskly stirred with a lightning or equivalent mixer at a slow to medium speed. Once mixing is concluded, any lumps that were present in the solution should preferably be evenly dispersed. No heat should be applied to this solution.

Once the solution comprised of the dry-blended mixture, the first liquid mixture and water is formed, if hydrogenated starch hydrolysate (HSH) or an active ingredient is to be added to the solution, it should be added at this step. A hydrogenated starch hydrolysate, such as HSH Stabilite SD30, may preferably be used as a bulking agent. SD30 is a hydrogenated starch hydrolysate in spray-dried form that is a low-sweetness powder and can be dissolved in water to produce clear, noncrystallizing syrups. When it is combined with water, the water must be maintained at room temperature in order to ensure adequate mixing and dispersement of the ingredient in solution. When incorporated into the bulking system contemplated by the present invention HSH preferably comprises less than 4% of the bulking system.

Active ingredients include, but are not limited to: vitamins; minerals; mineral salts; caffeine: dimethicone; pheobromine; central nervous system stimulants; amino acids; appetite suppressants; SSRIs; MAOI's; electrolytes; hydroxy citric acid; 5-hydroxy tryptophan (5-HTP); NSAids including acetaminophen, ibuprophen, aspirin or salicylic acid; glycerol; weight loss ingredients; and over-the-counter (OTC) medicines including, but not limited to, allergy/sinus medicines (such as diphenhydramine HCl), cough suppressants (such as dextromephorphan HBr), antihistamines, and nasal decongestants (such as pseudoephedrine HCl) may be added following addition of HSH, if desirable. These active ingredients, if incorporated into the bulking system, typically comprise approximately 3-4% of the resultant bulking system but may range from 0 to 7%. This system for delivery of active ingredients is useful for children as well as adults who would express a preference for ingesting these active ingredients in a soft candy form which may have a more pleasant taste and may be more enjoyable to consume than an active ingredient in pill form, for example.

After HSH and/or active ingredients are added, if desirable, to the solution comprised of the dry-blended mixture, the first liquid mixture, and water, the resultant mixture is stirred constantly to disperse any lumps in the mixture. After the mixture is fully dispersed, heat is preferably applied to the mixture to reach a first predetermined temperature of approximately 160 degrees Fahrenheit. Once this first predetermined temperature is reached, the mixture is held at that temperature for a period of time dependent on the type of equipment and amounts of ingredients used but preferably for approximately two minutes. While holding at the first predetermined temperature, the mixture is preferably stirred or agitated constantly.

After maintaining the first predetermined temperature for a set period of time, the heat beings applied to the mixture is preferably reduced to a low temperature in the range of 100 to 110 degrees Fahrenheit. After the heat has been reduced to this second predetermined temperature range, the second liquid mixture is added while stirring. Once the second liquid mixture is added, the resultant composition is held in the kettle or mixing tank for panning, molding or extruding. After holding in the kettle or mixing tank, the resultant composition is plated or molded in sprayed or prepared pans, allowed to cool, and then depanned. Alternatively, the resultant composition may be extruded and allowed to cool.

The process described above used to make the novel bulking system is devised so as to produce a product that will plate, mold and set faster than prior soft or hard candy products. Temperatures used when the product is at the stage for heating and molding should be kept to a maximum of 165 degrees Fahrenheit. Preferably, the soft or hard candy product should not remain in the mixer for more than two hours prior to heating or molding. Once the heating steps are complete, the product should be molded immediately and not left to stand in the mixer or in packaging equipment. Further, the components of the bulking system should not be left in a pre-measured or pre-weighed state for more than two hours because of the hydroscopic nature of the ingredients.

It is preferable that the setting temperature be maintained at approximately 160 degrees Fahrenheit. This setting temperature is important for loading active ingredients such as vitamins, over-the-counter (OTC) drugs, as well as acetaminophens (or NSAids) because high heat exposure potentially will destroy these active ingredients. The setting temperatures are quite cool in that the finished product gels and sets below 180 degrees Fahrenheit which is below the melting point of every NSAid currently on the market.

Finished products can include weight loss snacks and bars that can be either extruded or molded. When a reference is made to extrusion, this is usually when the product is squirted out and then cut, such as when licorice rope or bars are made. When molded, such as molding that takes place with gummy bears, the molds are pressed into cornstarch, the melted ingredients are poured in, and then are finished with a carnauba wax in a panning process. Weight loss/energy/meal replacement bars are extruded. In order to make a bulking system extrudable, one adds flour or a flour analog. Preferably, the flour or flour analog comprises 0 to 15% of the resultant bulking system. A type of flour that may be used is Konjac flour which has a high fiber content (such as 95%). The use of rice flour or starch are other possibilities. The preference is to use a flour that has no glutens. A soy milk powder also may be preferably included with the flour or flour analog to form an extrudable finished product, and if soy milk powder is used, it also preferably comprises 0 to 15% of the resultant bulking system.

Different types of finished product applications formed by the method described in the present invention include low-calorie gummy products, non-rolled fruit snacks, gummy products loaded with vitamins, energy-producing gummy products, gummy products for weight loss, chocolate chews, fruit extruded bar or rope/twist, as well as hard candies, bars, licorice ropes or analogs, fruit snacks, and rolled fruit snacks. Rolled fruit snacks are made by spraying a thin layer of mixed, molten liquid onto wax paper or some other paperlike substrate. Rolled fruit snacks or other fruit snack products, in the context of the described bulking system invention, contain additionally a low dextrose equivalent (low DE) fruit concentrate with returned fruit essence. While these types of bulking systems are specifically identified, it should be appreciated that other bulking systems may be produced by the process discussed in the context of this invention.

In one embodiment of the present invention, a soft candy product having a fruit flavor and loaded with ibuprophen is formed. Gelatin type B 250 bloom and pectin amid AF 020 are combined to form the dry-blended mixture. Polydextrose (or modified polydextrose) and erithritol are combined to form the first liquid mixture previously described. The second liquid mixture is preferably comprised of malic acid, fruit concentrate (such as strawberry or pear), color, flavors, sucralose and water. This second liquid mixture preferably comprises 0 to 5% of the resultant soft candy composition. The dry-blended mixture and the first liquid mixture are combined, and then HSH is added, followed by ibuprophen. Preferably ibuprophen comprises 0 to 7% of the resultant soft candy composition. Following combination of the above described ingredients (with the exception of the second liquid mixture), heat is applied to the solution to reach a first predetermined temperature of preferably 160 degrees Fahrenheit. The solution is held at this first predetermined temperature for a specified period of time, and then the heat is preferably reduced to a second predetermined temperature range of approximately 100 to 110 degrees Fahrenheit. When the second predetermined temperature range is reached, the second liquid mixture is added to the solution, and the composition that forms is held for later panning or molding.

Variations of the above described embodiment may be formed wherein, for example, acetaminophen is substituted for ibuprophen as the active ingredient. The dry-blended mixture as well as the first and second liquid mixtures remain at the same percentages of the resultant soft candy composition even with substitution of the active ingredient. Accordingly, the active ingredient typically will comprise 0 to 7% of the resultant soft candy composition.

While many of the embodiments described include malic acid as part of the second liquid mixture, it should be appreciated that lactic acid may be substituted for malic acid without appreciably changing the percent composition of the resultant bulking system. Lactic acid is preferably used when the bulking system is to have a brown flavor, meaning that the flavor has been typically derived from two basic thermal processes: caramelization and Maillard reactions. Brown flavors include, but are not limited to, chocolate, vanilla, toffee, mocha, cream/milk, cinnamon and caramel. If lactic acid is preferably used as part of the formulation of the bulking system, it should be appreciated that the colors and flavors incorporated into the second liquid mixture will be altered as fruit concentrate typically would not be utilized when a brown flavor is desired.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacturer compositions of matter, means, methods, or steps.

What is claimed is:

1. A method for producing a no-laxation bulking system, said method comprising:
blending a first set of ingredients comprising gelatin and pectin;
blending a second set of ingredients comprising polydextrose and erithritol;
blending a third set of ingredients comprising at least one acid and water;
adding said first set of ingredients to said second set of ingredients, forming a first mix;
adding water to said first mix and heating to a first predetermined temperature;
maintaining said first predetermined temperature for a predetermined time period; and
reducing heat to a second predetermined temperature and adding said third set of ingredients.

2. The method of claim 1 wherein no mixing or heating occurs when said first set of ingredients is added to said second set of ingredients.

3. The method of claim 1 wherein said heating to said first predetermined temperature occurs under constant agitation.

4. The method of claim 1 wherein said third set of ingredients is added while stirring constantly.

5. The method of claim 1, said method further comprising:
holding said combination of said first mix, water, and said third set of ingredients for panning, molding or extruding.

6. The method of claim 1, said method further comprising:
plating said combination of said first mix, water and said third set of ingredients in sprayed pans;
cooling said combination; and
depanning said combination.

7. The method of claim 1, said method further comprising:
adding hydrogenated starch hydrolysate (HSH) to said first mix, wherein said HSH is added prior to heating to said first predetermined temperature.

8. The method of claim 1, said method further comprising:
introducing an active ingredient to said first mix, wherein said active ingredient is added prior to heating to said first predetermined temperature.

9. The method of claim 8 wherein said active ingredient is selected from the group comprising: vitamins, minerals, mineral salts, caffeine, pheobromine, central nervous system stimulants, amino acids, appetite suppressants, SSRIs, MAOI's, electrolytes, hydroxy citric acid, 5-hydroxy tryptophan (5-HTP), NSAids, acetaminophen, ibuprophen, aspirin, salicylic acid, glycerol, weight loss ingredients, over-the-counter (OTC) medicines, allergy/sinus medicines, diphenhydramine HCl, cough suppressants, dextromephorphan HBr, antihistamines, nasal decongestants, and pseudoephedrine HCl.

10. The method of claim 1 wherein said third set of ingredients further comprises: a coloring agent, flavors, and sucralose.

11. The method of claim 1 wherein said at least one acid is selected from the group comprising:
malic acid and lactic acid.

12. A no-laxation bulking system, said bulking system comprising:
a dry-blended mixture comprising gelatin and pectin;
a first liquid mixture of polydextrose and erithritol, wherein said dry-blended mixture is added to said first liquid mixture forming a first combination without blending and water is introduced prior to heating said first combination; and
a second liquid mixture of at least one acid and water, wherein said second liquid mixture is added to said first combination.

13. The system of claim 12, said second liquid mixture further comprising:
color, flavors and sucralose.

14. The system of claim 12 wherein said polydextrose is selected from the group comprising:
refined polydextrose FCC, Litesse Ultra liquid, Litesse II and modified polydextrose.

15. The system of claim 12 further comprising:
a hydrogenated starch hydrolysate (HSH), wherein said HSH is added prior to heating said first combination.

16. The system of claim 12 further comprising:
an active ingredient, wherein said active ingredient is added prior to heating said first combination.

17. A no-laxation soft candy product, said soft candy product comprising:
a dry-blended mixture, said dry-blended mixture comprising 0 to 12% gelatin and 0 to 5% pectin;
a first liquid mixture, said first liquid mixture comprising 10 to 80% polydextrose and 2 to 12% erithritol;
a second liquid mixture, said second liquid mixture comprising 0 to 5% acid and water; and
water,
wherein said dry-blended mixture, said first liquid mixture, said second liquid mixture, and said water are combined.

18. The soft candy product of claim 17 wherein said dry-blended mixture is added to said first liquid mixture without blending to form a first combination.

19. The soft candy product of claim 18 wherein said water is added to said first combination prior to heating.

20. The soft candy product of claim 19 wherein said heat is reduced prior to adding said second liquid mixture.

21. The soft candy product of claim 17, said second liquid mixture further comprising:
color, flavor, and sucralose.

22. The soft candy product of claim 18, said soft candy product further comprising:
a hydrogenated starch hydrolysate (HSH) wherein said HSH is added prior to addition of said second liquid mixture to said first combination.

23. The soft candy product of claim 17, said soft candy product further comprising:
0 to 12% hydrogenated starch hydrolysate (HSH).

24. The soft candy product of claim 18, said soft candy product further comprising:
at least one active ingredient, wherein said at least one active ingredient is added prior to addition of said second liquid mixture to said first combination.

25. The soft candy product of claim 17, said soft candy product further comprising:
0 to 7% active ingredient.

26. The soft candy product of claim 17, said soft candy product further comprising:
flour or flour analogs to make said soft candy product extrudable.

27. The soft candy product of claim 17, said soft candy product further comprising:
0 to 15% flour or flour analog.

28. The soft candy product of claim 26 wherein said soft candy product is in bar form.

29. The soft candy product of claim 26 wherein said soft candy product is in rope form.

30. A no-laxation hard candy product, said hard candy product comprising:
a dry-blended mixture, said dry-blended mixture comprising 0 to 12% gelatin and 0 to 5% pectin;
a first liquid mixture, said first liquid mixture comprising 10 to 80% polydextrose and 2 to 12% erithritol;
a second liquid mixture, said second liquid mixture comprising 0 to 5% acid and water; and
water,
wherein said dry-blended mixture, said first liquid mixture said second liquid mixture, and water are combined.
